(19) United States
(12) Patent Application Publication
(10) Pub. No.: US 2017/0119290 A1
(43) Pub. Date: May 4, 2017

(54) METHOD AND APPARATUS OF NON-INVASIVE BIOLOGICAL SENSING USING CONTROLLED SUCTION DEVICE

(71) Applicants: Lujing Cai, Morganville, NJ (US); Amrish Patel, Holmdel, NJ (US)

(72) Inventors: Lujing Cai, Morganville, NJ (US); Amrish Patel, Holmdel, NJ (US)

(21) Appl. No.: 14/929,311

(22) Filed: Oct. 31, 2015

(51) Int. Cl. A61B 5/145; A61B 5/1477; A61B 5/00; A61B 5/1455

(52) U.S. Cl. CPC A61B 5/14532; A61B 5/1455; A61B 5/1477; A61B 5/6834; A61B 2560/0223; A61B 2560/0247

(57) ABSTRACT

The present invention relates to systems and methods using optical or electrical spectroscopy for accurate detection and monitoring of biological tissue properties in a noninvasive manner. To perform in vivo diagnose with more accurate and repeatable measurements, an air-tight micro suction cup is placed against biological tissue under test (such as skin of a patient), around which an electrical or optical sensing system comprising excitation and detection sensors is integrated. Applying a high power suction pump over the micro cup, a negative pressure is generated to reshape the skin covered by the cup to a contour suitable for better measurement results. Most important, as the suction power increases, certain amount of blood flow or body fluid is brought to skin layer, providing great potential of improving those diagnoses that require direct analysis over these biological components.

### BACKGROUND

Diabetes is a chronic disease which consists of various metabolic disorders. It is characterized by high levels of blood glucose and it is the result of a deficiency of insulin secretion or of resistance to the action of insulin or a combination of these [1]. Diabetes therapy must maintain near normal glycaemia values (60-120 mg/dl) in diabetic patients but this is so difficult that there is a need for a blood glucose monitoring system that can provide information throughout the day. Conventional blood sampling methods are painful and not able to monitor the glucose level continuously. In the last two decades, the non-invasive biological sensing has attracted extensive studies on techniques for evaluation of glycemia with an in vivo and noninvasive manner, i.e. techniques not requiring blood collection. The areas of studies vary in a wide range of different technologies, which can mainly be classified in two categories: optical sensing (Near/Mid infrared spectroscopy, and Raman/fluorescence spectroscopy[2][3][4][5]), or electrical sensing (bioelectrical impedance spectroscopy, dielectric analysis, and electromagnetic sensing [6][7][8][9]). In terms of accuracy and repeatability as compared to current existing blood-sample based method, however, noninvasive glucose monitoring have not yet reached to a mature stage for practical clinical application. The major difficulties stem from the fact that these technologies all have, because of the noninvasive nature, weaker access to the body issue that is directly correlated to the glycaemia content, such as blood.

### SUMMARY OF INVENTION

The present invention relates to systems and methods using optical or electrical spectroscopy for more accurate detection and monitoring of various biological tissue properties in a noninvasive or minimally invasive manner. The sensing system comprises an excitation source, and a single or a plurality of receiving sensors that collects the signal passing though the biological tissue under test, such as skin of human body. To perform in vivo diagnose with more accurate and repeatable measurements, an air-tight micro suction cup is placed facing against the skin of a patient, around which the sensing system is integrated. Applying a high power suction pump over the micro cup, a negative pressure is generated that reshapes the skin inside the cup to a contour suitable for better measurement results. More important, as the suction power increases, certain amount of blood flow or body fluid is brought to the skin layer, improving those diagnoses that require analysis over the blood or the body fluid.

### BRIEF DESCRIPTION OF THE DRAWINGS

The invention will be more fully understood by reference to the following drawings which are for illustrative purposes only:

FIG. 1 illustrates the non-invasive sensing system using micro suction cup, deployed with a single optical sensor;

FIGS. 2 and 3 describe detailed design of the suction cup system;

FIG. 4 presents operation procedures of the suction cup sensing system;

FIG. 5 illustrates the non-invasive sensing system using micro suction cup, deployed with a single electrical sensor;

FIG. 6 illustrates the non-invasive sensing system using micro suction cup, deployed with an array of optical sensors;

FIG. 7 is illustrates the non-invasive sensing system using micro suction cup, deployed with an array of electrical sensors;

FIG. 8 illustrates the non-invasive sensing system using micro suction cup, deployed with a single optical sensor, wherein a feedback to the suction pumping is applied to control the suction power to right amount;

FIG. 9 presents operation procedures of the suction cup sensing system with feedback to control the suction power;

FIG. 10 gives an example of the use of the thermal sensor in a multiple optical sensor suction cup sensing system with feedback;

FIG. 11 illustrates the non-invasive sensing system using micro suction cup, deployed with a single optical sensor, wherein a humility sensor is applied to improve the diagnoses accuracy;

FIG. 12 illustrates the non-invasive sensing system using micro suction cup, deployed with a single optical sensor, wherein an calibration path is applied to cope with the fluctuation caused by an unstable input;

FIG. 13 presents operation procedures of the suction cup sensing system when the calibration path and the humility sensor are deployed.

### DETAILED DESCRIPTION

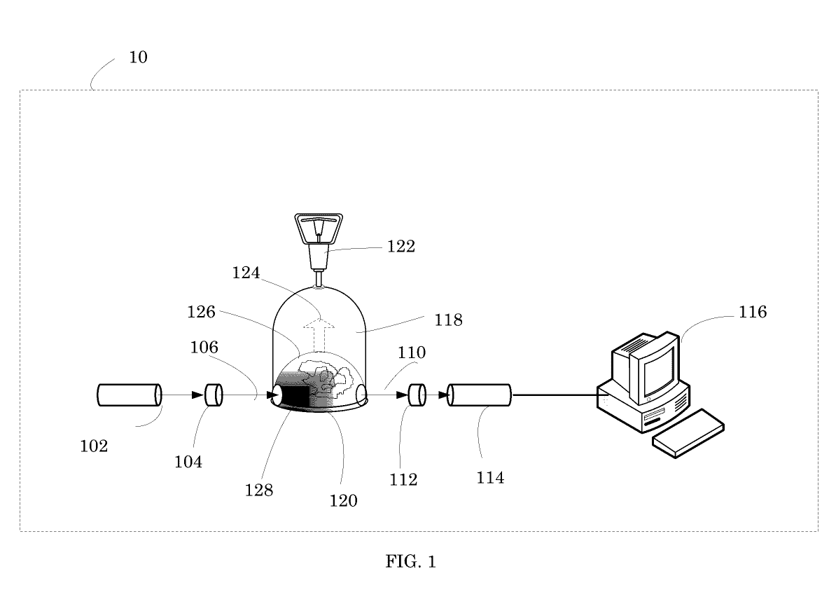

Referring more specifically to the drawings, for illustrative purposes the present invention is embodied in the methods and apparatus a generally shown in FIG. 1 through FIG. 13. It will be appreciated that the apparatus may vary as to configuration and as to details of the parts, and that the method may vary as to the specific steps and sequence, without departing from the basic concepts as disclosed herein.

Referring now to FIG. 1, a first non-invasive biological sensing system 10 is schematically described in accordance with the present invention. The device 10 comprises an excitation or transmitting source 102 coupled to a first light guide 104, such as a fiber optic unit, to direct and transport excitation light 106 to the biological tissue under measurement, such as skin 108 of a strategically selected area of the human body. The light signal 110 that is scattered from the tissue under test is collected and transported by a second light guide 112 to an receiving optical sensor (or light detector) 114, wherein the light signal is converted to electric signal and sent to the computer analysis unit 116. As means of improve measurement accuracy and repeatability, a micro suction cup 118, is placed between the light entry and exit points, with opening facing to the surface of the skin. The suction cup is made air tight by the seal ring 120. By connecting a high power air pump 122, the air inside the micro cup is pumped out, generating suction pressure 124 that raises up the skin surface 126 to a desirable contour. With sufficiently high suction pressure, the blood flow (or body fluid) 128 is brought to the skin layer, improving those diagnoses that typically require analysis over the blood or body fluid.

The suction pump 122 described in FIG. 1 performs suction to get the air out of the sealed cup. It can be mechanical operating manually, or electrical powered by an electrical motor.

The computer unit 116 described in FIG. 1 performs the analysis over the data collected from the receiving sensor and delivers the diagnose result. The analysis may include near infrared spectral imaging, Raman or fluorescence spectroscopy, or other type of optical based spectral analysis methods.

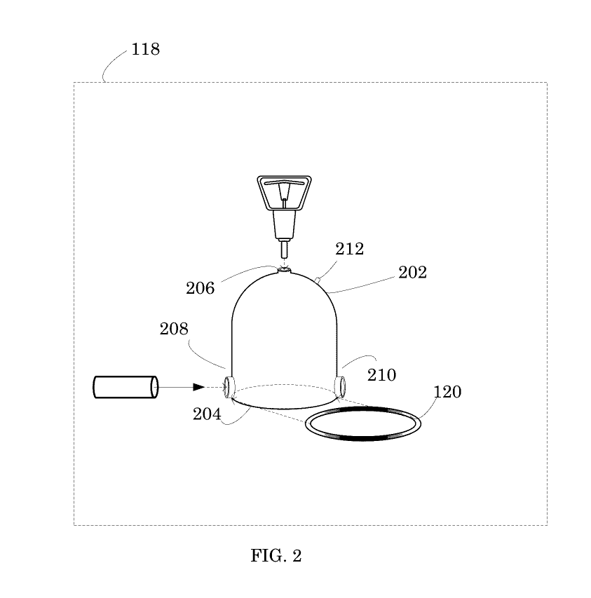

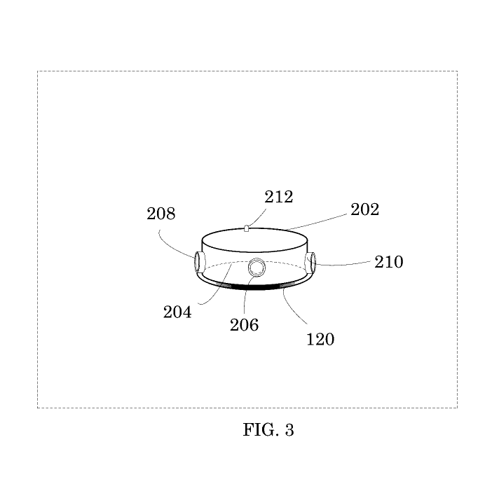

Referring to FIG. 2, the micro suction cup 118 in FIG. 1 further comprises a cup-shaped capsule 202 with an opening 204, which is placed against the sample under test. The capsule can be typically made of glass, plastic, steel or other solid materials. A seal ring 120, which is typically made of rubber or silicon, or other type of sealing materials, is attached to the opening to keep air-tight between the skin and the capsule. There is also small opening 206 to connect to the air suction pump. Located at bottom of the cup, closing to the skin, 208 and 210, are used to connect the excitation source and receiving sensor respectively. Wherever devices are connected to the capsule, air-tight seal needs to be maintained at each of the openings. A release valve 212 is placed somewhere on the cup to release the suction when the measurement is complete. The micro suction cup device can be shaped differently to meet custom needs. For example, FIG. 3 shows a design of shadow cylinder shape to reduce the size, where the block 202,204, 206,208,210, and 120 have the same functionality as in FIG. 2.

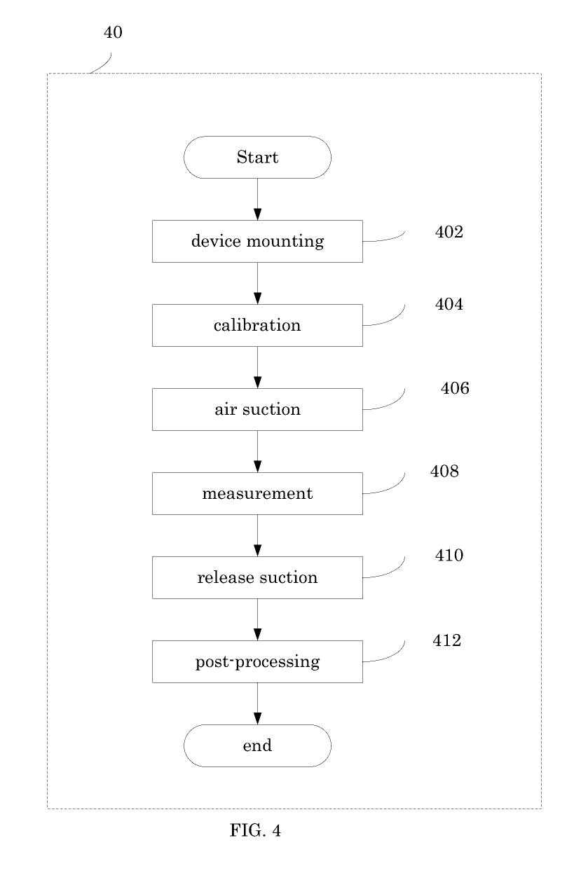

FIG. 4 describes the procedure of operating the cup sensing system in an in vivo manner. In a first step 402 of the procedure, the micro cup is properly mounted at the selected skin area of interest. The cup opening with seal ring (120 in FIG. 1) has to be placed against the skin with sufficient tightness so that air-tight sealing is ensured. In case of non-flat skin area at some locations, the opening shape can be specially made to follow the contour of human body. In a second step 404 of the procedure, the excitation source, receiving sensor, and computer unit (102, 114, 106 in FIG. 1) are all turned on. When all these units reach steady state ready for operation, the computer unit starts to collect certain amount of data for calibration purpose. The computer may also optionally give an indication if the cup sensing system is appropriately mounted. In a third step 406 of the procedure, the suction pump is activated. When skin inside the cup is raised up to a desired level because of the suction, the suction pump is stopped. In a fourth step 408 of the procedure, the computer starts to collect data while the skin level inside the cup is maintained. In a fifth step 410 of the procedure, the release valve (212 in FIG. 2) is pressed to release the suction when sufficient data is taken by the computer unit. In a sixth step 412 of the procedure, the computer unit performs analysis over the data collected in step 408. The data captured during calibration step 404 may be optionally used to improve the diagnose outcome.

FIG. 4 specifies the operation of the biological sensing system in more precise and detailed manner, it is understood, however, one or some steps may be optionally omitted and the order of the execution of the steps may vary in actual implementation.

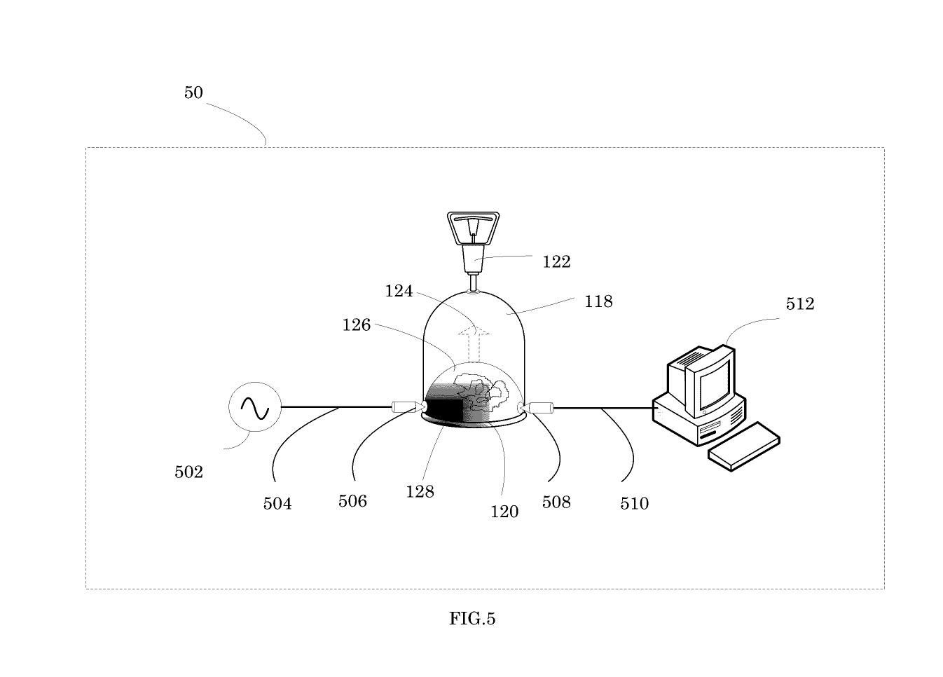

Referring now to FIG. 5, a second non-invasive biological sensing system 50, which is using the electrical sensors, is schematically described in accordance with the present invention. The device 50 comprises an electrical excitation source 502 connected to, via an electrical wire 504, to an excitation probe 506. The excitation source 502 can typically generate electrical signals such as DC, AC, impulse, or other types of signal waveforms. The excitation probe 506 is passing through the small opening of the micro suction cup 118 (described in FIG. 2) to be directly in touch with the skin area 126 under test. At the other opening of the micro suction cup 118, a sensing probe 508 is installed, which is connected, via electrical wire 510, to the computer analysis unit 512. The computer unit 512 therefore can process the electrical signal received, such as providing impedance or dielectric analysis. The rest items in FIG. 5, including 118,120,124,126 and 128, can be similarly described as in FIG. 1 and FIG. 2, and the corresponding operation procedure also follows the steps given in FIG. 4.

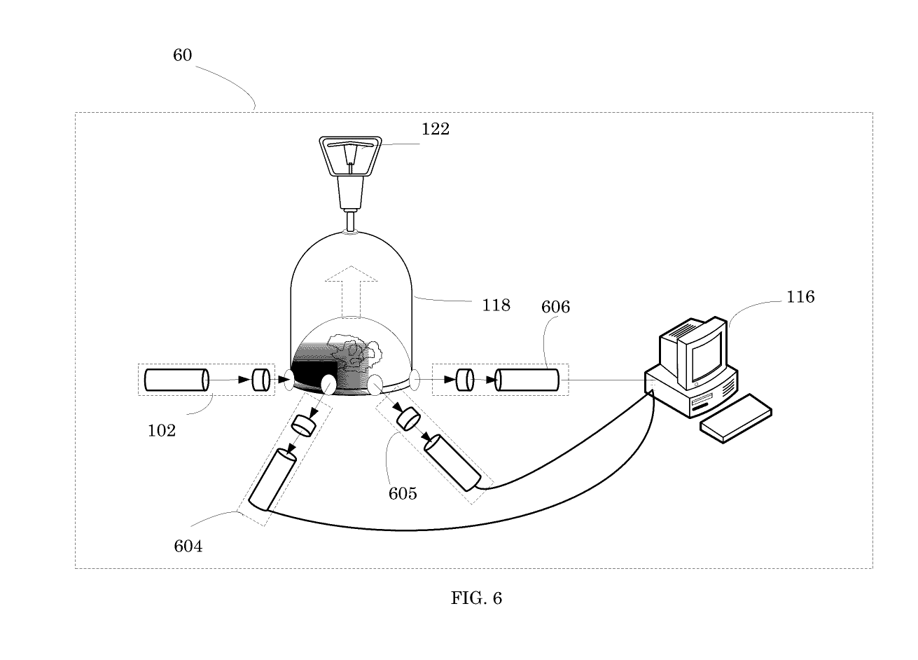

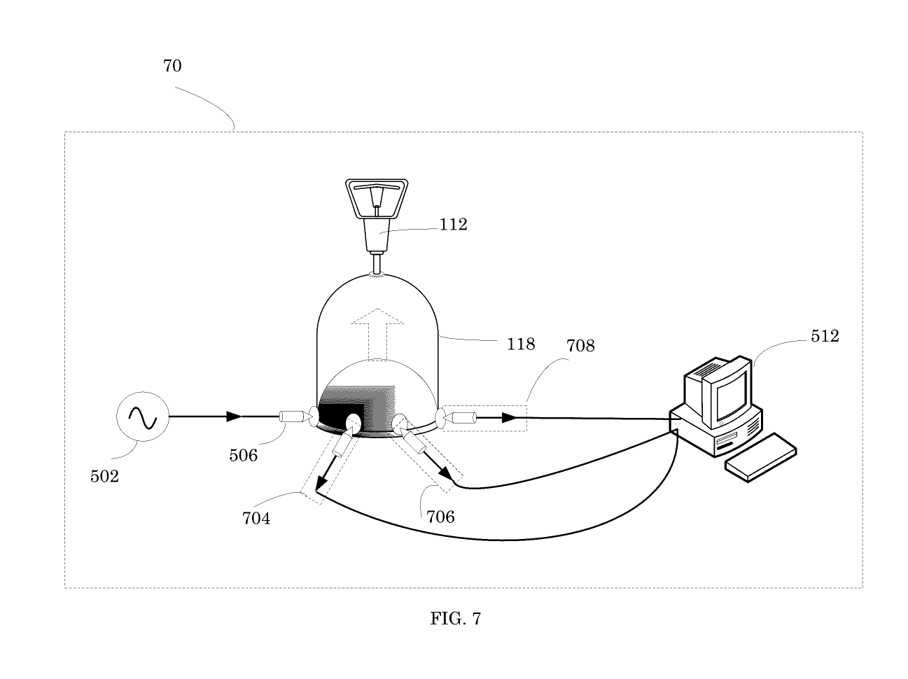

The micro cup sensing system can be extended to a design of using sensor arrays, where a plurality of receiving sensors deployed around the cup generate a plurality of signals sent to the computer unit in order to improve the diagnose accuracy. FIG. 6 and FIG. 7 show such designs using optical and electrical sensors respectively.

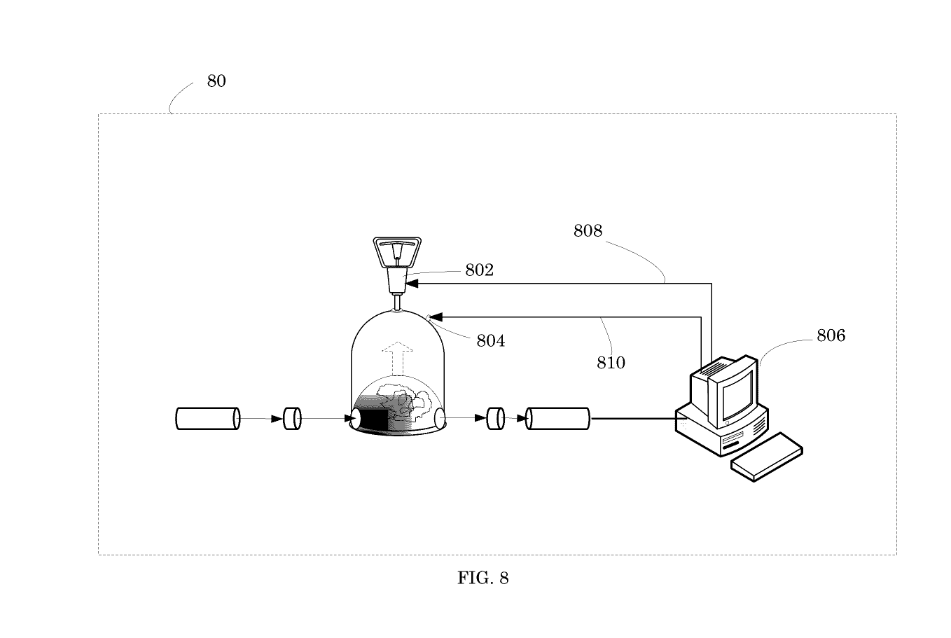

Referring to FIG. 8, a third non-invasive biological sensing system 80, which is using a feedback control over the air pressure inside the suction cup, is schematically described in accordance with the present invention. The system 80 comprises a first link 808 that connects the computer unit 806 to the suction pump 802, and second link 810 that connects the computer unit 806 to the release valve 804. Based on the measurement results, the computer unit dynamically controls (such as switch on and off) the suction pump and the release valve via the feedback links, in order to achieve optimal pressure inside the suction cup for best diagnose results. As an option, the release valve should be manually controlled to override suction process anytime during the feedback procedure. As another option, a pressure sensor can be installed inside the suction cup and connected to the computer unit, in order to accurately control the suction pressure to a desired level. Note that FIG. 8 shows an example of the feedback suction cup system using optical sensors. It should be directly applicable to other variations of the designs described in this invention disclosure, such as the sensor systems using electrical sensors, or using sensor arrays.

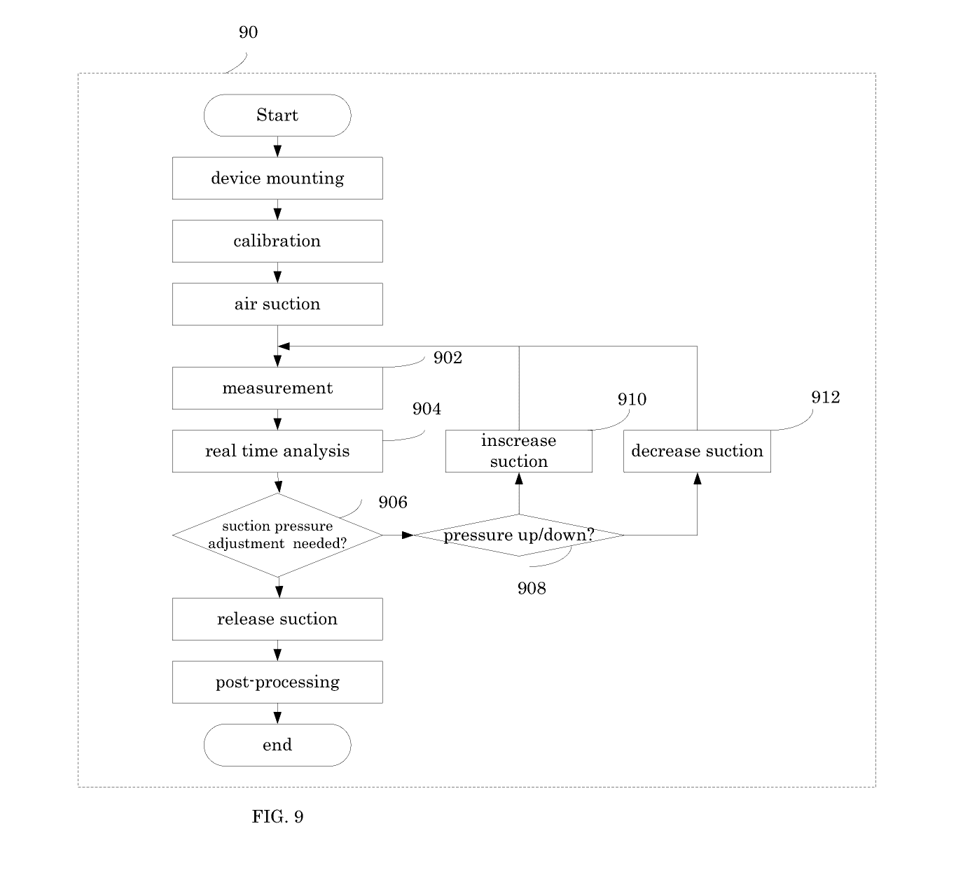

An operation procedure 90 of the suction feedback control is shown in FIG. 9. The procedure is essentially based on all the steps described in FIG. 4, except that additionally steps are added for the feedback control of the suction pressure. In a first additional step 902, the receiving sensor collects measurement data received from the tissue under test after the air suction step. In a second additional step 904, a real time analysis 904 over the measured data is performed by the computer unit 806 referred in FIG. 8. In a third additional step 906, the computer unit decides whether suction pressure adjustment is need based on the analysis results. If decision is no, the procedure will proceed further steps including release the suction and post processing, using similar steps described in FIG. 4. Otherwise, the computer unit will further decide whether the suction pressure needs be increased or decreased in a fourth additional step 908. For increasing the suction pressure, the computer unit, via the feedback link 808 in FIG. 8, switches on the suction pump in a fifth step 910 for a controlled time period, and for decreasing the suction pressure, the computer unit, via the feedback link 810, partially opens the release valve in a sixth additional step 912 for another controlled time period. After these steps, the measurement is retaken in step 902 and the aforementioned step cycle repeats until decision is made by the computer unit that no further suction pressure adjustment is needed. As an option of the first additional step 902, the measurement data is taken from an optional pressure sensor installed inside the suction cup and the third additional step 906 makes decision based on the measured pressure level, in order to precisely control the suction pressure to a desired value.

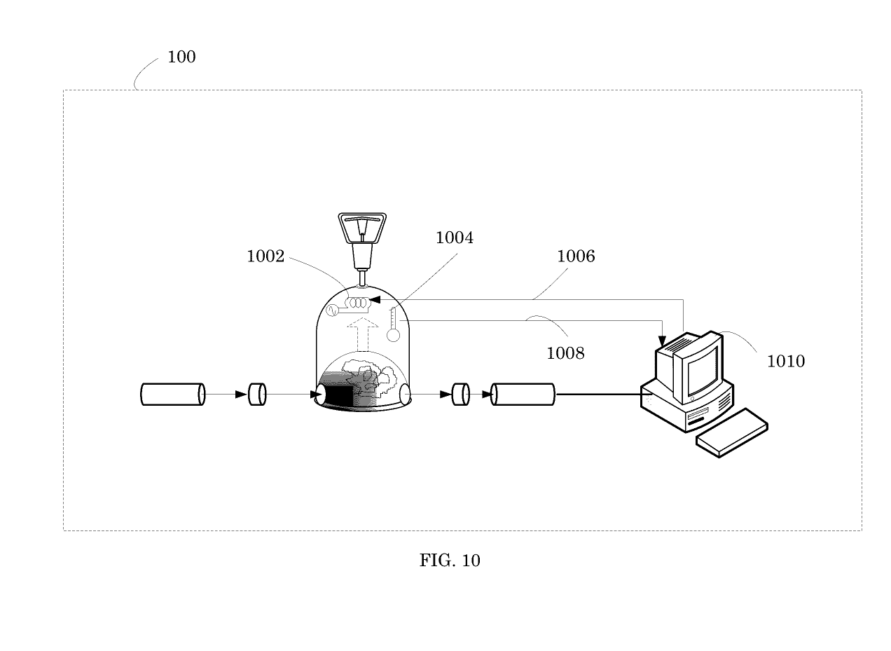

Referring FIG. 10, a heating device 1002 is deployed inside the suction cup to raise the air temperature for different diagnose purposes. A thermal meter 1004 can also be installed inside the suction cup for controlling the temperature. Both 1002 and 1004 are controlled by the computer unit 1010 via the wire connections 1006 and 1008. Clearly, a temperature adjustment by a feedback procedure can be designed to maintain the temperature to a desired value.

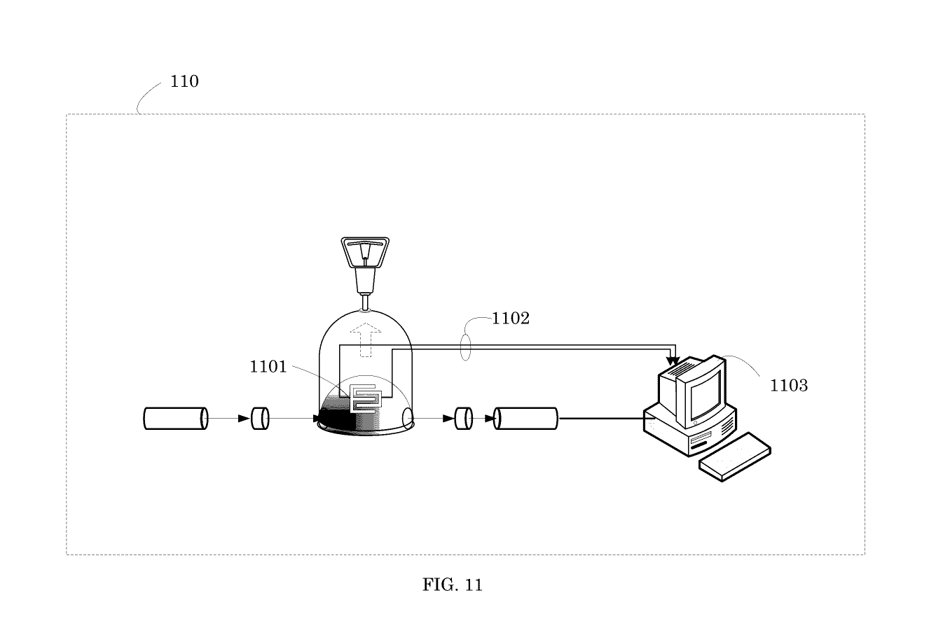

Referring FIG. 11, a humility sensor 1101 is deployed inside the suction cup to measure the humility of the skin under test. The humility sensor 1101 is placed tightly against the surfaced of the tissue under test, such as skin of a human body, in order to provide analysis of its water contents. This humility sensor is connected to the computer unit 1103 via the wire 1102. In FIG. 11, an interdigital-shaped humility sensor is applied as an example. Other types of humility sensor can also be utilized.

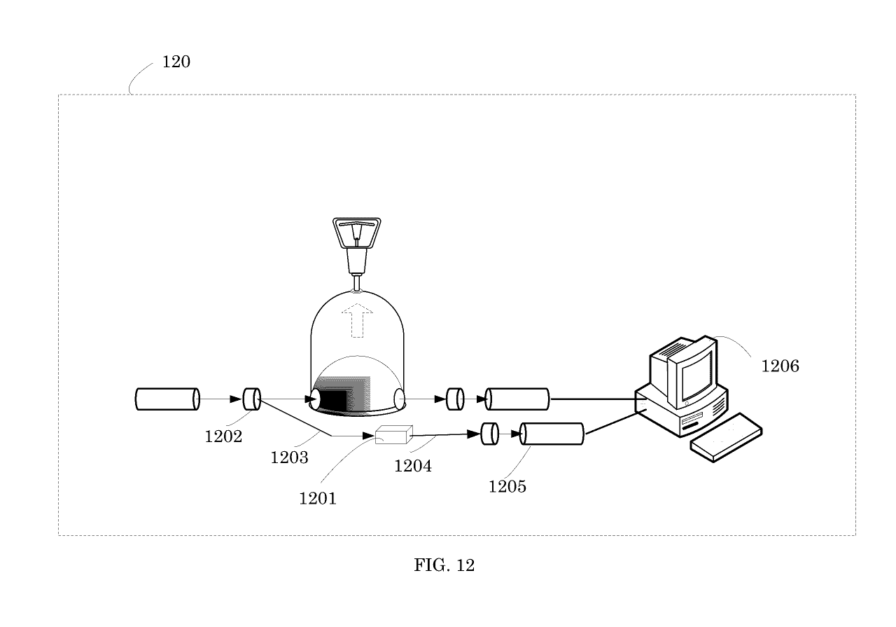

Referring FIG. 12, an auxiliary sensor 1204, which can be either optical or electrical, is deployed to the suction cup sensing system for the purpose of calibration. This sensor also receives the signal emitted by the excitation source 1202, passing through an attenuator 1201 connected by links 1203 and 1204. The attenuator 1201 is made of the material of known and constant optical or electrical property. For example, the attenuator has a known optical absorption coefficient for any of the optical sensing systems described in the disclosure. The auxiliary sensor 1204 further feeds the detected signal to the computer unit 1206 for processing.

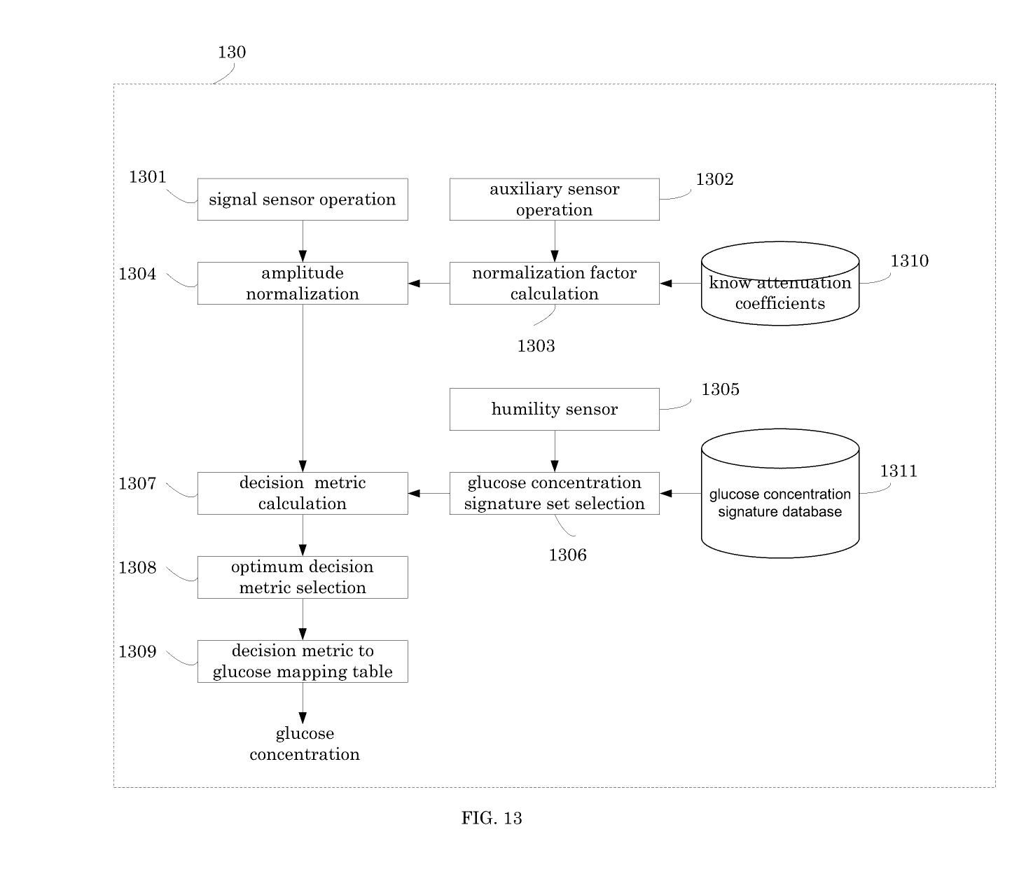

FIG. 13 describes the procedure 120 whereas the humidity sensor and the calibration auxiliary sensor are applied to any of the biological glucose sensing systems described in this embodiment. In a first step 1301 of the procedure, the main receiving sensor that is connected to skin under test in the suction cup, collects a spectrum of signals (which can be either optical or electrical) at different wavelengths or frequencies, and send it to the computer unit of the sensing system. In a second step 1302 of the procedure, the auxiliary sensor collects second spectrum of signals and sends it to the computer unit. Though described in two separate steps, the step 1301 and 1302 occurs concurrently when the excitation source described in any of the sensing systems in this embodiment emits at given frequencies or wavelengths. In a third step of the procedure 1303, a compensation factor is calculated from the input of the auxiliary sensor and the attenuation coefficients stored in the computer at each frequency or wavelength point. As a simple example, assume Sa(f) is the input from the auxiliary detector and the R(f) as the constant attenuator coefficient at frequency f, the compensation factor C(f) is calculated by C(f)=|Sa(f)|/R(f), whereas |Sa(f)| represents the amplitude of signal from the auxiliary sensor. Other non-linear algorithm can also applied to calculated C(f). In a fourth step of the procedure, the signal from the main sensor is normalized by the compensation factor calculated by 1303. In a fifth step 1305 of the procedure, the humility sensor senses the humility of the skin and sends the signal also to the computer unit. This step can occur before or after, or same time as, the step 1301. In a sixth step of the procedure 1206, the computer unit selects a set of reference signature from the database 1311 based on the sensed humility level. This set of signature consists of a series of reference spectrum calibrated beforehand in terms of various concentration level of the biological tissue property under measurement, such as glucose. In a seventh step of the procedure 1307, the computer unit correlates the received spectrum with the selected reference signature set and calculates a plurality of decision metrics, corresponding to each member of the reference signature set. As an example, the correlation algorithm can be partial linear regression (PLR) or principle component regression (PCR). In a eighth step of the procedure 1308, a single metric is selected among the calculated decision metrics according to a optimum criterion, such as a maximum rule or any other nonlinear algorithm. At last step 1206 of the procedure, this selected decision metric is uniquely mapped to the concentration level of the measured biological tissue property from a pre-calibrated table.

FIG. 13 specifies the operation of the calibration and humility sensors in a general manner, it is understood, however, one or some steps may be optionally omitted and the order of the execution of the steps may vary in actual implementation.

The apparatus and methods disclosed in the entire embodiment of this invention are mainly focusing on noninvasive blood glucose sensing and monitoring via a suction cup sensing system directly applied to skin of a human body. It will be more appreciated, however, that the same methods and techniques can be directly applied to sensing and monitoring other types of biological tissue properties that partially needs in-depth access to the body tissue, such as blood. Examples of the biological tissue properties may be some clinically important blood analytes such as albumin, cholesterol, or urea.

### REFERENCES

[1] American Diabetes Association, (2010) “Diagnosis and Classification of Diabetes Mellitus”, Diabetes Care, vol. 33, pp. S62-S66.
[2] David D. Cunningham2 and Julie A. Stenken, “Near-Infrared Spectroscopy for Noninvasive Glucose Sensing”, Chapter 13, In Vivo Glucose Sensing, Volume 174, 357-390, 2010 John Wiley & Sons, Inc.
[3] Syed M. Ali, Franck Bonnier, and etc. “Raman spectroscopic analysis of human skin tissue sections ex-vivo: evaluation of the effects of tissue processing and dewaxing”, Journal of Biomedical Optics 18(6), 061202 (June 2013).
[4] Karthik Vishwanath and Nimmi Ramanujam, “Fluorescence spectroscopy in vivo”, Encyclopedia of Analytical chemistry, 2011 John Wiley & Sons, Ltd.
[5] David C. Klonoff, M.D., FACP, “Overview of Fluorescence Glucose Sensing: A Technology with a Bright Future”, Journal of Diabetes Science and Technology, Volume 6, Issue 6, 1242-1250, November 2012
[6] Tushar Kanti Bera, “Bioelectrical Impedance Methods for Noninvasive Health Monitoring: A Review”, Journal of Medical Engineering, Volume 2014, Article ID 381251, http://dx.doi.org/10.1155/2014/381251.
[7] M. Gourzi, A. Rouane, R. Guelaz, M. S. Alavi, M. B. McHugh, M. Nadi, et al., “Noninvasive glycaemia blood measurements by electromagnetic sensor: study in static and dynamic blood circulation, J. Med. Eng. Technol. 29(2005)22-26.
[8] V. Pockevicius, V. Markevicius, “Blood Glucose Level Estimation Using Interdigital Electrodes”, ELEKTRONIKA IR ELEKTROTECHNIKA, ISSN 1392-1215, VOL. 19, NO. 6, 2013, http://dx.doi.org/10.5755/j01.eee.19.6.4566.
[9] Tura A., Maran A., Pacini G., (2007) “Non-invasive glucose monitoring: Assessment of technologies and devices according to quantitative criteria”, Science Direct, vol. 77, pp. 16-40.

What is claimed is:

1. An apparatus of improving measurement accuracy of optical or electrical spectroscopy for detection and monitoring of various biological tissue properties in a noninvasive or minimally invasive manner, comprising:
a suction system that reshape the contour of the samples under test; and
a excitation source that transmit signals to the sample under test; and
a receiving sensor, or plurality of receiving sensors that collect the signal passing through the sample under test; and
a computer unit that performs analysis and diagnose.

2. The apparatus of claim 1, wherein the suction system includes a micro cup and a suction pump.

3. The system of claim 2 wherein the micro suction cup comprising:
an opening attached with a seal ring, which makes air tight between the micro suction cup and the sample under test; and
an small opening that is used to connect to the suction pump; and
a plurality of openings that are used to connect to the excitation and receiving sensors; and
a release valve that is used to reduce the suction pressure inside the suction cup.

4. The system of claim 2, wherein the suction pump may operate manually by a pumping handle, or automatically by an electric motor.

5. The device of claim 4, wherein the electric motor may be switched on or off by the computer unit to form a feedback control.

6. The device of claim 3, wherein the release valve can be controlled by the computer unit.

7. The apparatus of claim 1 wherein further comprising a heater in order to raise the temperature inside, and a thermal meter to sense the temperature.

8. The device of claim 7, wherein the heater can be control by the computer unit.

9. The apparatus of claim 1 wherein further comprising a humidity sensor in order to monitor the humidity of the biological tissue under test.

10. The device of claim 9, wherein the humidity sensor is connected to the computer unit.

11. The apparatus of claim 1, wherein further comprising following devices for calibration purpose:
an auxiliary receiving sensor; and
an attenuator of known electrical or optical property

12. The devices in claim 11, wherein comprising configurations:
the attenuator is directly connected to the excitation source and auxiliary sensor; and
the auxiliary sensor is connected to the computer unit.

13. An method of improving measurement accuracy of optical or electrical spectroscopy for detection and monitoring of various biological tissue properties in a noninvasive or minimally invasive manner, comprising:
reshaping contour of the biological tissue under test by a suction system; and
measuring the signals passing through the tissue under test by transmitting from a excitation source and receiving from a receiving sensor, or plurality of receiving sensors; and
performing control and analysis by a computer unit.

14. The method of claim 13, wherein the operation is further characterized by following steps:
mounting the suction cup against tissue under test and making the attachment air tight; and
calibrating the sensing system by making initial measurement without starting the suction pump; and
activating the suction pump and stopping it when the sample under test in the suction cup has achieved the desired contour; and
collecting measurement data by the computer unit; and
activating the releasing valve to release the suction.

15. The method of claim 14, wherein the releasing valve is additionally controlled by following feedback steps
performing real time analysis by computer unit after collecting the measurement data; and
deciding if suction pressure adjustment is needed from the analysis result; and
partially opening the release valve if deciding to reduce the suction pressure; or
activating the suction pump if deciding to increasing the suction pressure; and
collecting the measurement data and performing the real time analysis again; and
repeating the feedback steps until desired analysis result is achieved; and
performing final post processing analysis by the computer unit.

16. The method of claim 13, wherein operation of the auxiliary sensor is characterized by following steps:
collecting the first measurement data from the main receive sensor by the computer unit; and
collecting simultaneously the second measurement data from the auxiliary sensor by the computer unit; and
calibrating the first measurement data using the calibration factor calculated by the second measurement data; and
performing post analysis by the computer unit.

17. The method of claim 13, wherein operation of the humidity sensor is characterized by following steps:
collecting the first measurement data from the main receive sensor by the computer unit; and
collecting the second measurement data from humidity sensor and calculating humidity level; and
selecting the a set of reference signatures based on the sensed humility level; and
correlating the first measurement data with the selected reference signature to calculated a plurality of decision metrics; and
selecting a single decision metric from the calculated decision metrics according to a optimum criterion rule; and
mapping the selected decision metric to a level of biological tissue property being measured based on a pre-calibrated table.
